(19) United States
(12) Patent Application Publication
(10) Pub. No.: US 2004/0241100 A1
(43) Pub. Date: Dec. 2, 2004

(54) Nasally administrable compositions of zolpidem and methods of use

(75) Inventors: Stephen J. Kramer, Houston, TX (US); Louis F. Fabre, Houston, TX (US)

Correspondence Address: OBLON, SPIVAK, MCCLELLAND, MAIER & NEUSTADT, P.C., 1940 DUKE STREET, ALEXANDRIA, VA, 22314, US

(73) Assignee: Fabre Kramer Pharmaceutical, Inc., Houston, TX (US)

(21) Appl. No.: 10/801,856

(22) Filed: Mar. 17, 2004

Related U.S. Application Data
(60) Provisional application No. 60/454,619, filed on Mar. 17, 2003.

(51) Int. Cl. A61L 9/04; A61K 31/4745

(52) U.S. Cl. 424/45; 514/303

(57) ABSTRACT

One embodiment of the present invention provides a pharmaceutical composition for nasal administration, which includes zolpidem, a prodrug thereof, a pharmaceutically acceptable salt thereof, or a combination thereof, and a pharmaceutically acceptable nasal carrier in liquid form. Another embodiment of the present invention provides a method for inducing sleep, which includes nasally administering to a subject in need thereof a pharmaceutical composition, which includes zolpidem, a prodrug thereof, a pharmaceutically acceptable salt thereof, or a combination thereof, and a pharmaceutically acceptable nasal carrier in liquid form.

### BACKGROUND OF THE INVENTION

1. Field of the Invention

This invention relates to compositions and methods for the nasal administration of zolpidem and pharmaceutically acceptable salts thereof.

2. Discussion of the Background

Zolpidem is known to possess anxiolytic, anti-anoxic, sleep-inducing, hypnotic and anticonvulsant properties; and it is useful for the treatment of anxiety states, sleep disorders and other neurological and psychiatric complaints, for the treatment of vigilance disorders, in particular for combating behavioral disorders which can be attributed to cerebral vascular damage and to the cerebral sclerosis encountered in geriatrics, and also for the treatment of epileptic vertigo due to cranial trauma and for the treatment of metabolic encephalopathies. In addition to the treatment of insomnia, zolpidem has been claimed to be useful in the treatment of other conditions such as convulsions (U.S. Pat. No. 4,382,938), migraine headaches (U.S. Pat. No. 5,767,117), and Parkinsonian and related extrapyramidal symptoms (WO 96/31210). The entire contents of the aforementioned references are hereby incorporated by reference.

Zolpidem, chemically named N,N,6-trimethyl-2-(4-methylphenyl)-imidazo{1,2-a}pyridine-3-acetamide, is a non-benzodiazepine hypnotic. Zolpidem has the following structure:

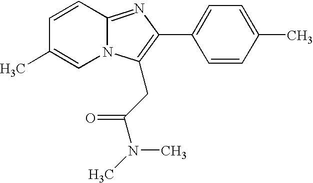

A 2:1 tartrate complex of zolpidem has the chemical name N,N,6-trimethyl-2-(4-methylphenyl)-imidazo{1,2-a}pyridine-3-acetamide hemitartrate, and is sold under the tradename AMBIEN. The hemitartrate (sometimes referred to hereinafter as “tartrate”) is indicated for the short-term treatment of insomnia. Physicians' Desk Reference, 2979-2983 (57^{th} ed. 2003), incorporated herein by reference. The synthesis of zolpidem is described in, for example, U.S. Pat. Nos. 4,382,938 and 4,794,185, incorporated herein by reference.

Zolpidem is a hypnotic agent having a chemical structure unrelated to benzodiazepines, barbiturates, and other drugs with known hypnotic properties, but it interacts with a GABA-BZ receptor complex and shares some of the pharmacological properties of benzodiazepines. The sedative, anticonvulsant, anxiolytic, and myorelaxant drug properties of zolpidem are believed to derive from its ability to allosterically modulate the activity of GABA_{A} complexes by increasing trans-membrane conductance of chloride ions. The major modulatory site of the GABA_{A} receptor complex is located on its alpha (α) subunit and is referred to as the benzodiazepine (BZ) or omega (ω) receptor. At least three subtypes of the (ω) receptor have been identified.

Zolpidem is readily absorbed through the gastrointestinal tract, and is reportedly eliminated almost entirely in the liver—largely by oxidation of the methyl groups on the phenyl and imidazopyridine rings to the corresponding carboxylic acids. Goodman & Gilman's The Pharmacological Basis of Therapeutics, Hardman, J. G., et al., eds., 372 (9^{th} ed., 1996), incorporated herein in its entirety by reference. Metabolism of zolpidem is primarily attributed to CYP3A4, and it has been found that inducers of CYP3A4 such as rifampicin, phenyloin, and carbamazepine reduce the pharmacodynamics of zolpidem. Villikka, K., et al., Clin. Pharmacol. Ther. 62(6):629-634 (1997), incorporated herein by reference.

The metabolites of zolpidem are reportedly inactive. See, e.g., Goodman & Gilman 's The Pharmacological Basis of Therapeutics, Hardman, J. G., et al., eds., 372 (9^{th} ed., 1996); Physicians' Desk Reference, 2930 (53rd ed. 1999), the entire contents of each of which being hereby incorporated by reference.

U.S. Pat. No. 6,333,345, incorporated herein by reference, discloses that while zolpidem is effective in the treatment of insomnia, there are adverse effects associated with both short term and chronic use of the drug including headache, dizziness, vertigo, confusion, lack of coordination, lethargy or drowsiness the day after use, and gastrointestinal problems such as nausea and diarrhea.

U.S. Pat. No. 5,603,943, incorporated herein by reference, discloses a nasally administrable composition, which includes a powdery or crystalline polyvalent metal carrier having a mean particle size of not more than 250 μm. Zolpidem is disclosed but not exemplified. Such a composition is difficult to meter and use.

U.S. Pat. No. 5,929,061 and U.S. Pat. No. 5,767,117, both incorporated herein by reference, disclose methods and compositions for treating vascular headaches using benzodiapines that bind to GABA_{A}. Zolpidem is disclosed as a benzodiazepine useful for treating migraines.

U.S. Pat. Re. No. 36,744, incorporated herein by reference, discloses compositions and methods for nasal administration of benzodiazepine hypnotics. Zolpidem is neither disclosed nor suggested.

### SUMMARY OF THE INVENTION

Accordingly, one object of the present invention is to provide a way to administer zolpidem, which is efficient and easy for the patient to use.

Another object of the present invention is to provide a way to administer zolpidem, which reduces the need for supervision of administration.

Another object of the present invention is to provide a way to administer zolpidem, which minimizes or bypasses first pass metabolism.

Another object of the present invention is to provide a way to administer zolpidem, which has a beneficial pharmacokinetic profile.

Another object of the present invention is to provide a way to administer zolpidem, which is superior to conventional routes of administration.

These and other objects have been achieved by the present invention, the first embodiment of which provides a composition for nasal administration, which includes zolpidem, a prodrug thereof, a pharmaceutically acceptable salt thereof, or a combination thereof, and a pharmaceutically acceptable nasal carrier in liquid form.

Another embodiment of the present invention provides a method for inducing sleep, which includes nasally administering to a subject in need thereof a composition, which includes zolpidem, a prodrug thereof, a pharmaceutically acceptable salt thereof, or a combination thereof, and a pharmaceutically acceptable nasal carrier in liquid form.

### DESCRIPTION OF THE PREFERRED EMBODIMENTS

Various other objects, features and attendant advantages of the present invention will be more fully appreciated as the same becomes better understood from the following detailed description of the preferred embodiments of the invention, which is not intended to be limiting unless otherwise specified.

A preferred embodiment of the invention provides a composition for nasal administration, which includes a sleep-inducing amount of zolpidem, a pharmaceutically acceptable salt thereof, or a combination thereof, and a pharmaceutically acceptable nasal carrier in liquid form.

Another preferred embodiment of the present invention provides a method for inducing sleep, which includes nasally administering to a subject in need thereof a composition, which includes a sleep-inducing amount of zolpidem, a pharmaceutically acceptable salt thereof, or a combination thereof, and a pharmaceutically acceptable nasal carrier in liquid form.

The dosage amount is suitable to obtain a sleep-inducing amount or systemic, therapeutically effective amount of active ingredient and may preferably range from about 0.001 mg to about 250 mg of active ingredient per day, given as a single once-a-day dose or as divided doses from 2, 3 or 4 times per day. These ranges include all values and subranges therebetween, including 0.003, 0.005, 0.007, 0.009, 0.01, 0.03, 0.05, 0.07, 0.09, 0.1, 0.3, 0.5, 0.7, 0.9, 0.2, 1, 2, 3, 4, 5, 6, 7, 8, 9, 10, 15, 20, 25, 30, 35, 40, 45, 50, 75, 100, 150, 200, 225, and 250 mg and any combination thereof.

The drug unit volume is suitable to provide a sleep-inducing amount or systemic, therapeutically effective amount of active ingredient to a subject in need thereof, and the drug unit may preferably range from 0.001 ml to 4 ml in volume. This range includes all values and subranges therebetween, including 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.13, 0.15, 0.17, 0.19, 0.2, 0.21, 0.23, 0.25, 0.27, 0.29, 0.31, 0.33, 0.35, 0.37, 0.39, 0.4, 0.5, 0.7, 0.9, 1, 1, 2, 3 and 4 ml, and any combination thereof.

The preferred weight/weight loading of the active ingredient of the invention compositions range from 0.01 to 95% by weight, based on the total weight of the composition. These ranges include all values and subranges therebetween, including 0.03, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 40, 60, 80, 85, 90, 91, 92, 93, and 94%, and any combination thereof.

One preferable embodiment of the invention provides a pharmaceutical composition, which includes zolpidem, a pharmaceutically acceptable salt thereof, or a combination thereof, and a pharmaceutically acceptable nasal carrier in liquid form, and optionally one or more therapeutic ingredients known to those skilled in the art.

The compositions of the invention include, but are not limited to solutions, emulsions, microemulsions, nanoemulsions, dispersions, suspensions, elixirs, drops, sprays, aerosols, syrups, gels, ointments, and the like. Preferably, the composition is in liquid form at room temperature (20-25° C., which range includes 20, 21, 22, 23, 24 and 25° C.) at standard pressure.

Optionally, the composition may be frozen, however, and in the form of a solid for storage and then thawed to a liquid immediately prior to use.

The composition may also be in a liquid form under pressure in a pressurized container or non-pressurized container and released in the form of an aerosol or spray during administration.

As used herein, nasal administration includes contacting the composition with the nasal mucosa. Optionally, the composition may be administered using a nasal tampon or a nasal sponge containing a composition of the present invention.

The active ingredient may be suitably combined with a pharmaceutically acceptable nasally administrable carrier according to conventional pharmaceutical compounding techniques. These techniques include but are not limited to dissolving, dispersing, suspending, spray-drying, mixing, sonicating, ultrasonicating, emulsifying, microemulsifying, nanoemulsifying, and the like.

The composition can be sterilized, for example, by filtration through a bacterial-retaining filter, or by incorporating sterilizing agents in the form of sterile solid compositions which can be dissolved or dispersed in sterile water or other sterile medium prior to use.

The formulation of the invention may contain any further chemical entity or any association of two or more chemical entities.

Some examples of and/or components of the nasally administrable carrier include one or more of the following, alone or in any combination: water; saline solutions; pyrogen-free water; isotonic saline; Ringer's solution; alcohols such as ethanol; isopropanol; 1,3-butanediol; glycols such as propylene glycol; glycol ethers such as polyethylene glycol; glycerin, sorbitol, mannitol; vegetable, or mineral oils such as mineral oil, light mineral oil, peanut oil, cottonseed oil; safflower oil; sesame oil; olive oil; corn oil; soybean oil; arachis oil, groundnut oil, germ oil, castor oil; sunflower oil; hydrogenated vegetable oil; synthetic or naturally occurring mono-, di- or triglycerides; surfactants; polysorbates; sugars such as lactose, glucose and sucrose; starches, corn starch, potato starch, sodium starch glycolate, tapioca starch, pre-gelatinized starch; cellulose and its derivatives such as sodium carboxymethyl cellulose, ethyl cellulose and cellulose acetate; calcium carboxymethyl cellulose; polyvinyl pyrrolidone, methyl cellulose, hydroxypropyl methyl cellulose, microcrystalline cellulose, microcrystalline cellulose, croscarmellose sodium, crospovidone, polacrilin potassium; powdered tragacanth; malt; gelatin; talc; cocoa butter, wax; ethyl oleate ester and ethyl laurate ester; fatty acid esters of sorbitan; talc, calcium carbonate (e.g., granules or powder), microcrystalline cellulose, powdered cellulose, dextrates, kaolin, silicic acid, pre-gelatinized starch, agar; magnesium hydroxide; aluminum hydroxide; alginic acid; fatty acids, fatty acid salts, oleic acid, stearates, palmitates, sodium lauryl sulfate, magnesium stearate, calcium stearate, stearic acid, zinc stearate; diluents such as for example water or other solvents; solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, glycerol, tetrahydrofurfuryl alcohol, polyethylene glycols and fatty acid esters of sorbitan; natural and synthetic gums such as acacia, sodium alginate, other alginates, agar, powdered tragacanth, guar gum; coloring agents; releasing agents; sweeteners; flavoring agents; perfuming agents; preservatives and antioxidants. Combinations are possible.

Other preferable compounds which may be included in the composition include mucoadhesives such as chitosan hydroxycellulose, hyaluronic acid, carbopol 934P and 947P; penetration agents or enhancers such as tween 80, hydroxy bile salts, and pyroglutamates; and preservatives such as benzalkonium chloride. Combinations are possible. Such compounds, and others, are disclosed in U.S. Pat. Nos. 3,836,665 and 4,789,667, the entire contents of each of which being hereby incorporated by reference.

Another form includes a buffer for the nasal delivery system, which can be selected from the group including acetate, citrate, prolamine, carbonate and phosphate buffers. The pH of the buffer may be selected to maintain the active ingredient in an ionized or non-ionized form. Suitable buffering agents can be selected such that when the formulation is delivered into the nasal cavity of a mammal, selected pH ranges are achieved therein upon contact with, e.g., the nasal mucosa. The buffer pH may also be suitably selected to enhance absorption of the active ingredient across the nasal mucosa of the subject.

Preferably, the buffer is selected such that the composition has a pH of from about 3 to about 10, which range includes all values and subranges therebetween, including 3.5, 3.6, 3.7, 3.8, 4, 4.5, 5, 5.1, 5.2, 5.3, 5.4, 5.5, 5.6, 5.7, 5.8, 5.9, 6, 6.1, 6.2, 6.3, 6.4, 6.5, 6.7, 6.8, 6.9, 7, 7.1, 7.2, 7.3, 7.4, 7.5, 7.6, 7.7, 7.8, 7.9. 8, 8.1, 8.2, 8.3, 8.4, 8.5, 9 and 9.5. Combinations of buffers are possible.

In the formulation of the present compositions, a relatively water soluble form of the active ingredient may be employed. Use of a fully dissolved form of the active ingredient maximizes its immediate effect. Compositions containing the active ingredient in a form having a limited solubility may be employed where sustained release is desired. These compositions, in which the active ingredient is not totally solubilized in its dosage form provide a prolonged therapeutic activity. For this purpose, a long chain carboxylic acid salt of the active ingredient is possible. The acid portion of the salt preferably contains from about 10 to about 30 carbon atoms. Such salts, including stearates, palmitates and the like, are readily synthesized by known techniques.

The term “pharmaceutically acceptable salt” is well known in the art, as described in S. M. Berge et al. (J Pharmaceutical Sciences, 66: 1-19, 1977), incorporated herein in its entirety by reference. Suitable pharmaceutically acceptable salts include salts of the active ingredient prepared from pharmaceutically acceptable non-toxic organic or inorganic acids. Preferred examples of suitable non-toxic acids include, but are not limited to, maleic, fumaric, benzoic, ascorbic, embonic, succinic, oxalic, bis-methylenesalicylic, methanesulfonic, ethanedisulfonic, acetic, propionic, tartaric, salicylic, citric, gluconic, lactic, mandelic, cinnamic, citraconic, aspartic, stearic, palmitic, itaconic, carbonic, perchloric, malonic, glycolic, p-amino-benzoic, glutamic, benzene sulfonic and theophylline acetic acids, 8-halotheophyllines such as 8-bromo-theophylline, hydrochloric, hydrobromic, sulfuric, sulfamic, phosphoric, nitric, alginic, anthranilic, camphorsulfonic, ethenesulfonic, formic, furoic, galacturonic, glucuronic, isethionic, malic, mucic, pamoic, pantothenic, phenylacetic, propionic, sulfanilic, p-toluenesulfonic acid. A preferred non-toxic acid is tartaric acid. Combinations are possible.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, furnarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pictate, pivalate, propionate, stearate, succinate, sulfate, tartrate, thiocyanate, p-toluenesulfonate, undecanoate, valerate salts, and the like.

Other excipients and/or compounds which may be preferably included in the composition are described in “REMINGTON'S PHARMACEUTICAL SCIENCES”, 14th edition, 1970; and “HANDBOOK OF PHARMACEUTICAL EXCIPIENTS”, 3^{nd} edition, Arthur H. Kribbe, ed., 2000, the entire contents of each of which being incorporated by reference.

Most preferably, the carrier is or includes one or more of water, sterile purified water USP, sterile water for inhalation USP, saline or isotonic saline. Most preferably, the zolpidem is in the form of a 2:1 tartrate salt, such as in AMBIEN. Most preferably, the composition is an aqueous solution. Preferably, the patient or subject is a mammal and most preferably a human.

Another embodiment of the invention provides a pharmaceutical composition which contains one or more prodrugs of zolpidem and a pharmaceutically acceptable nasal carrier in liquid form. Known prodrugs of zolpidem are incorporated herein by reference.

Other embodiments of the invention include treating and/or preventing a disease or condition selected from the group including sleep disorder, insomnia, affective disorder, depression, attention deficit disorder, attention deficit disorder with hyperactivity, attention deficit/hyperactivity disorder, convulsive disorders, epilepsy, anxiety, acute anxiety, chronic anxiety, aggressive behavior, spasticity, acute muscle spasm, behavioral disorder, schizophrenic disorder, mood anxiety, abnormal plasma hormone level associated disorder, endocrine disorder, vascular headache, migraine headache, and combinations thereof.

Most preferably, the disease or condition is sleep disorder and/or insomnia.

Preferably, inducing sleep is meant to include the treatment or prevention of sleep disorders and/or reducing the severity of symptoms associated with sleep disorders such as insomnia, insomnia of a primary nature with little apparent relationship to immediate somatic or psychic events, and insomnia which is secondary to some acquired pain, anxiety or depression. Symptoms associated with sleep disorders include, but are not limited to, difficulty in sleeping and disturbed sleep patterns.

The magnitude of a prophylactic or therapeutic dose of zolpidem in the acute or chronic management of the diseases or conditions recited herein may vary with the nature and severity of the disease or condition. The dose, and optionally the dose frequency, will further vary according to the age, body weight, and response of the individual patient. Suitable dosing regimens can be readily selected by those skilled in the art with due consideration of such factors and given the teachings herein.

It is possible to use dosages of the active ingredient outside the ranges disclosed herein in some cases. For example, a daily dose may be reduced by about 50% in elderly patients. Further, because elimination of zolpidem from the bloodstream is dependant on renal and liver function, it is contemplated that the total daily dose may be optionally and suitably reduced by about 75% in patients with moderate hepatic impairment, and that it may be reduced by about 50% in patients with mild to moderate renal impairment. It is noted that combined with the teachings herein the clinician or treating physician will know how and when to interrupt, adjust, or terminate therapy in conjunction with individual patient response. In addition, the clinician or treating physician would readily be able to determine which subject is in need of administration of the invention composition.

The efficacy of a drug is determined in part by its concentration in the blood of the subject being treated. In general, activity is dependent upon the bioavailability of therapeutic agent evidenced by that concentration. It is therefore desirable that the present nasal administration of active ingredients enables a significantly faster onset and more pronounced blood concentration than conventional forms of administration. This insures an elevated and more constant effect.

Like the benzodiazepines, zolpidem interacts with a GABA-BZ receptor complex and shares some of the pharmacological properties of benzodiazepines. Zolpidem, however, has a chemical structure unrelated to benzodiazepines, and, in contrast to the benzodiazepines, which nonselectively bind to and activate all omega receptor subtypes, zolpidem in vitro preferentially binds the (ω₁) receptor with a high affinity ratio of the (α₁/(α₅) subunits. The (ω₁) receptor is found primarily on the Lamina IV of the sensorimotor cortical regions, substantia nigra (pars reticulata), cerebellum molecular layer, olfactory bulb, ventral thalamic complex, pons, inferior colliculus, and globus pallidus. Without wishing to be bound by theory, this selective binding of zolpidem on the (ω) receptor, though not absolute, may explain the relative absence of myorelaxant and anticonvulsant effects in animal studies as well as the preservation of deep sleep (stages 3 and 4) in human studies of zolpidem at hypnotic doses.

Accordingly, one preferred embodiment of the provides a method for inducing sleep in a subject in need thereof, which includes nasally administering a composition including zolpidem, a prodrug thereof, a pharmaceutically acceptable salt thereof, or a combination thereof, and a pharmaceutically acceptable nasal carrier in liquid form to the subject, wherein the zolpidem, a prodrug thereof, a pharmaceutically acceptable salt thereof, or a combination thereof preferentially and/or selectively binds a (ω₁) receptor in the subject with a high affinity ratio of (α₁)/(α₅) subunits in the subject. The high affinity ratio is a relative term understood by those of ordinary skill in the art, and preferably represents the preferential binding of zolpidem as compared to the nonselective binding of a (non-zolpidem) benzodiazapine to the omega receptor subtypes.

Preferably, the composition is suitably nasally administered to a subject in need of sleep inducement just before bedtime, and preferably after a meal.

One embodiment of the invention encompasses a composition that does not include physiologically acceptable polyvalent metal carriers having a particle size of not more than. 250 μm. Another embodiment of the invention encompasses a composition which is substantially free of all mono- or di-saccharide excipients. Another embodiment encompasses a composition which is free of lactose. Another embodiment of the invention provides a composition which contains zolpidem in an amount less than its maximum solubility.

The entire contents of each of the aforementioned references, patents and applications are incorporated herein by reference, the same as if set forth at length.

Obviously, numerous modifications and variations of the present invention are possible in light of the above teachings. It is therefore to be understood that within the scope of the appended preferred embodiment, the invention may be practiced otherwise than as specifically described herein.

1. A pharmaceutical composition for nasal administration, comprising:
zolpidem, a prodrug thereof, a pharmaceutically acceptable salt thereof, or a combination thereof, and a pharmaceutically acceptable nasal carrier in liquid form.

2. The composition according to claim 1, wherein the composition is a solution of a 2:1 zolpidem/tartrate salt in a least one liquid medium selected from the group consisting of sterile purified water USP, sterile water for inhalation USP, saline, isotonic saline, and combinations thereof.

3. The composition according to claim 1, further comprising at least one pH buffer.

4. The composition according to claim 1, further comprising at least one penetrating agent.

5. The composition according to preferred embodiment claim 1, wherein the zolpidem is present in an amount ranging from 0.001 mg to about 250 mg.

6. A method for inducing sleep, comprising:
nasally administrating to a subject in need thereof a pharmaceutical composition comprising zolpidem, a prodrug thereof, a pharmaceutically acceptable salt thereof, or a combination thereof, and a pharmaceutically acceptable nasal carrier in liquid form.

7. The method according to claim 6, wherein the composition is a solution of a 2:1 zolpidem/tartrate salt in a least one liquid medium selected from the group consisting of sterile purified water USP, sterile water for inhalation USP, saline, isotonic saline, and combinations thereof.

8. The method according to claim 6, wherein the composition further comprises at least one pH buffer.

9. The method according to claim 6, further comprising at least one penetrating agent.

10. The method according to claim 6, wherein the active agent is present in an amount ranging from 0.001 mg to about 250 mg.

11. The composition according to claim 1, wherein the drug unit volume of active agent ranges from 0.001 ml to 4 ml.

12. The composition according to claim 1, wherein the weight/weight loading of active agent ranges from 0.01% to 95% by weight based on the total weight of the composition.

13. The composition according to claim 1, wherein the composition is in the form of a solution, an emulsion, a microemulsion, a nanoemulsion, a dispersion, a suspension, an elixir, drops, sprays, aerosols, syrups, gels or ointments.

14. The composition according to claim 1, wherein the composition is buffered to a pH of 3 to 10.
